(19) United States
(12) Patent Application Publication
(10) Pub. No.: US 2022/0400977 A1
(43) Pub. Date: Dec. 22, 2022

(54) LAPAROSCOPIC BOWEL LENGTH INDICATING DEVICES AND METHODS OF USE

(71) Applicant: Boehringer Technologies, LP, Phoenixville, PA (US)

(72) Inventors: Evan Langdale, Schwenksville, PA (US); Christopher L. Radl, Malvern, PA (US); Steven Moulden, West Chester, PA (US); Tahlia Detweiler, Perkiomenville, PA (US)

(21) Appl. No.: 17/834,171

(22) Filed: Jun. 7, 2022

Related U.S. Application Data
(60) Provisional application No. 63/211,155, filed on Jun. 16, 2021.

(51) Int. Cl. A61B 5/107; A61B 90/00; A61B 17/29; A61B 1/00; A61B 1/313

(52) U.S. Cl. CPC A61B 5/1076; A61B 90/06; A61B 17/29; A61B 1/00087; A61B 1/00101; A61B 1/3132; A61B 2090/061

(57) ABSTRACT

A measuring device and methods of use for measuring a section of a bowel of a patient. The measuring device includes a tubular section having a longitudinal axis and at least one wing normally projecting outward transverse to the longitudinal axis. The tubular section is configured for mounting on a conventional or robotic laparoscopic grasper adjacent the grasper's movable jaws. The at least one wing is are pivotable to a closed position so that grasper with the measuring device mounted thereon can be inserted through a conventional trocar, whereupon the at least one wing projects outward transversely to the longitudinal axis. The at least one wing is of a predetermined length to serve as a measurement tool enabling a surgeon to measure off a desired length of the bowel.

### CROSS REFERENCE TO RELATED APPLICATIONS

This non-provisional utility application claims the benefit under 35 U.S.C. § 119(e) of U.S. Provisional Patent Application Ser. No. 63/211,155 filed on Jun. 16, 2021 and entitled Laparoscopic Bowel Length Indicating Devices And Methods Of Use, the entire disclosure of which is incorporated by reference herein for all purposes.

### FIELD OF THE INVENTION

This invention relates generally to medical devices and methods and more particularly to devices and methods for laparoscopically measuring the length of an internal anatomic structure, e.g., the bowel, of a patient.

### SPECIFICATION

### Background of the Invention

Numerous laparoscopic and robotic surgical procedures require measurement of the small bowel. Bowel length can be especially important in certain bariatric surgeries, where the bowel is rerouted, and the length of the newly created bowel limbs is crucial in these standardized procedures. In bariatric procedures such as SADI-S and Roux En Y, if the bowel functional length is too short, known as short bowel syndrome, the patient will not absorb enough nutrients as food passes through, leading to malnutrition and if the bowel is left too long, there will not be a significant amount of weight loss and the surgery will have been ineffective, possibly resulting in a revision surgery.

Measuring bowel in surgery is not as simple as rolling out the bowel and using a standard measuring tape because the bowel is “raveled” in the abdomen and distances measured can be up to 3 meters and sometimes more. To measure these distances in a laparoscopic/robotic surgery, the surgeon “walks” the bowel with his/her graspers, beginning at an anatomical landmark and passing bowel between graspers in grasper-to-grasper steps, each of a fixed increment (usually 5 or 10 cm increments), while counting the number of increments (grasper-to-grasper steps) until the desired length has been reached.

While the above prior art procedure may be generally suitable for its intended purpose it nevertheless leaves much to be desired from the standpoint of accuracy and ease of measurement. In this regard some surgeons estimate these grasper-to-grasper distances by eye, not directly measuring each increment. This method can result in incorrect bowel length determination. Other surgeons utilize some external reference, such as a suture measured to a desired increment length or ruler. This method can result in increased time because for each increment counted, there is an extra step of holding the bowel up to the remote measurement reference.

Though it is well known that accurate measurement is crucial, currently there is no widespread consensus on the best means of doing so. When measuring lengths up to 300 cm, estimating each increment based purely on eyesight can introduce compounding error and result in larger error. Using an external reference still introduces some slight error but more importantly it can be time consuming to have to hold the bowel up to the reference with every pass.

Thus, a need exists for a device which is simple in construction and easy to use to effectively laparoscopically measure a length of bowel. The subject invention addresses that need by providing devices and methods for enabling surgeons to accurately and quickly measure the bowel, or other internal anatomic structures, without interrupting the current laparoscopic surgical technique.

### BRIEF SUMMARY OF THE INVENTION

One aspect of this invention is a measuring device for measuring internal anatomy in the body of a patient. The measuring device is configured for mounting on a laparoscopic grasper or being an integral component of the laparoscopic grasper. The laparoscopic grasper has an elongated shaft having a longitudinal axis and pair of grasping jaws located at a distal end of the shaft. The measuring device comprises at least one elongated wing and a retention structure. The at least one elongated wing has a predetermined length as measured along a longitudinal axis of the at least one elongated wing. The at least one elongated wing is configured for orientation in a state extending generally parallel to the longitudinal axis to enable the at least one elongated wing to be introduced through a trocar or some other port into the body of the patient. The at least one elongated wing is also configured for orientation in a state extending transverse to the longitudinal axis. The retention structure is configured for holding the at least one elongated wing on the elongated shaft of the laparoscopic grasper when the measuring device is mounted on the laparoscopic grasper and the at least one elongated wing is located within the body of the patient, whereupon when the at least one elongated wing is in the orientation extending transverse to the longitudinal axis the at least one elongated wing can be used to measure a length of the internal anatomy of the patient.

In accordance with one preferred aspect of the measuring device, the state extending generally parallel to the longitudinal axis comprises a collapsed or closed state and the state extending transverse to the longitudinal axis comprises a extended or open state. The measuring device additionally comprises a pivotable hinge biased to normally hold the at least one elongated wing in the open state. The pivotable hinge is also configured to be pivoted in response to an applied force to pivot the at least one elongated wing to said closed state, whereupon when said at least one elongated wing is in the closed state the measuring device can pass through a trocar or some other port into the body of the patient and when the measuring device is in the body of the patient the pivotable hinge automatically pivots the at least one elongated wing to the open state so that the at least one elongated wing can be used to measure a length of the internal anatomy of the patient.

In accordance with another preferred aspect of this invention the measuring device is configured for releasable mounting of the laparoscopic grasper. The measuring device additionally comprises a body portion configured to receive a distal portion of the laparoscopic grasper to releasably secure the measuring device on the laparoscopic grasper at a desired position.

In accordance with another preferred aspect of this invention the body portion comprises tubular sidewall including a central passageway, a longitudinally extending axis, a distal end and a proximal end. The tubular sidewall has an outer periphery. The central passageway has an inner surface configured to receive a distal portion of the shaft of the laparoscopic grasper to releasably secure the measuring device on the grasper at a desired position, and wherein the at least one wing is located at the distal end of the tubular sidewall.

In accordance with one preferred aspect of this invention the at least one wing comprises a pair of wings, each of which is pivotable between the open state and the closed state, and vice versa. Each of the wings of the pair of wings when in the closed state extend parallel to each other and to the longitudinal axis of the shaft to enable the laparoscopic grasper with the measuring device mounted thereon to be introduced through the trocar into the body of the patient, whereupon the pair of wings automatically pivot to the open state, wherein the wings extend in opposite radial directions transverse to the longitudinal axis of the shaft. Each of the pair of wings has a predetermined length to serve as measuring tool to enable the user to measure a length of the internal anatomy of the patient.

In accordance with one preferred aspect of this invention the laparoscopic grasper includes a peripheral portion configured to be engaged by a portion of the measuring device, and wherein the central passageway includes a retention structure configured to frictionally engage the peripheral portion of the laparoscopic grasper to hold the measuring device in place on the laparoscopic grasper at a desired position and is resistant to displacement from the desired position. The desired position is such that when the wings are in the open state the grasping jaws of the laparoscopic grasper are exposed and able to open and close as desired.

In accordance with one preferred aspect of this invention each of the wings of the pair of wings is connected to the tubular sidewall by a respective living hinge.

In accordance with another preferred aspect of this invention the body portion and the pair of wings constitute a molded one-piece member.

In accordance with another preferred aspect of this invention the retention structure includes a necked-down section located intermediate the proximal end and the distal end.

In accordance with another preferred aspect of this invention the retention structure includes an elastic ring located at the proximal end. The elastic ring provides additional friction for holding the measuring device at the desired position.

In accordance with another preferred aspect of this invention the retention structure includes a pair of slots in the necked-down section and extending parallel to the longitudinal axis. The slots facilitate stretching of the necked down section.

In accordance with another preferred aspect of this invention the retention structure includes a helical spring in the necked-down section. The helical spring is configured to provide additional friction for holding the measuring device at the desired position.

In accordance with another preferred aspect of this invention the retention structure comprises a front section of the central passageway. The front section tapers in a distal direction and includes a pair of slots extending parallel to the longitudinal axis from the distal end of the sidewall to a position adjacent the proximal end of the sidewall.

In accordance with another preferred aspect of this invention an internal diameter of the front section of the central passageway at the distal end is less than an external diameter of the distal end of the laparoscopic grasper, wherein the distal end of the laparoscopic grasper is configured to be introduced into the central passageway from the proximal end of the tubular sidewall and out of the central passageway at the distal end of the tubular sidewall. The pair of slots is configured to splay apart to enable the distal end of the laparoscopic grasper to exit out of the central passageway at the distal end of the tubular sidewall, with portions of the inner surface of the central passageway frictionally engaging portions of the periphery of the laparoscopic grasper.

In accordance with another preferred aspect of this invention the proximal end of the measuring device comprises a loop configured to be grasped by the jaws of a first laparoscopic grasper to enable the measuring device to be mounted on a distal end of a second laparoscopic grasper extending into the body of the patient.

In accordance with another preferred aspect of this invention the loop is configured to be grasped by the jaws of the first laparoscopic grasper by the second laparoscopic grasper to dismount the measuring device from the first laparoscopic grasper.

In accordance with another preferred aspect of this invention the measuring device comprises an integral component of a laparoscopic grasper.

Another aspect of this invention is a method of laparoscopically measuring internal anatomy of a patient comprising providing a first and second laparoscopic grasper, each laparoscopic grasper having a shaft and movable jaws located at a distal end of the shaft. A measuring device is provided for releasable mounted on the first laparoscopic grasper. The measuring device has a longitudinal axis and comprises at least one wing having a predetermined length, a proximal end and a distal end. The measuring device is mounted on a distal end of the first laparoscopic grasper with the jaws of the first laparoscopic grasper extending distally beyond the at least one wing. A second laparoscopic grasper is introduced into the body of the patient via a trocar. The first laparoscopic grasper is moved to a first position wherein one of the ends of the at least one wing is located at a starting point. The jaws of the first laparoscopic grasper are caused to grasp the internal anatomy at the first position. The second laparoscopic grasper is moved to a second position immediately adjacent one of the ends of the at least one wing and the pair of jaws of the second laparoscopic grasper is caused to grasp the internal anatomy at the second position. The jaws of the first laparoscopic grasper are caused to release the internal anatomy at the first position and the first grasper is moved to a third position wherein one of the ends of the at least one wing is located at the second position. The jaws of the first laparoscopic grasper are caused to grasp the internal anatomy at the third position. If desired, the movements of the first and second laparoscopic graspers are repeated a desired number of times and the number of movements of the first laparoscopic grasper is counted to measure a desired length of the internal anatomy.

In accordance with one preferred aspect of the method of this invention the measuring device comprises a pair of elongated wings whose distal ends extend opposite each other and transverse to the longitudinal axis, wherein when the first laparoscopic grasper is in the first position the distal end of one of the pair of wings is located at the starting point, and the distal end of the other of the pair of wings is located at the second position. When the first laparoscopic grasper is in the third position the distal end of one of the pair of wings of the first laparoscopic grasper is located at the third position, and the distal end of the other of the pair of wings is located at a fourth position. The method additionally comprises causing the jaws of the second laparoscopic grasper to be opened to release the second laparoscopic grasper from the internal anatomy and moving the second grasper to the fourth position.

In accordance with another preferred aspect of the method of this invention the measuring device comprises a single elongated wing extending transversely to the longitudinal axis when the first laparoscopic grasper is in the first position. The proximal end of the at least one wing is located at the starting point and the distal end of the at least one wing is located at the second position. When the first laparoscopic grasper is in the third position the proximal end of the at least one wing is located at the third position, and the distal end of the at least one wing is located at a fourth position. The method additionally comprises causing the jaws of the second laparoscopic grasper to be opened to release the second laparoscopic grasper from the internal anatomy and moving the second laparoscopic grasper to the fourth position.

In accordance with another preferred aspect of the method of this invention the at least one wing is pivotable between an open state and a closed state, and vice versa. The at least one wing is normally biased into the open state wherein the at least one wing extends transversely to the longitudinal axis, the at least one wing is collapsible to the closed state by a force applied thereto wherein the at least one wing extends parallel to the longitudinal axis, and wherein the method comprises causing the at least one wing to be in the closed state to enable the measuring device to be introduced laparoscopically into the body of the patient via a trocar, whereupon once within the body of the patient the at least one wing automatically pivots to the open state.

In accordance with another preferred aspect of the method of this invention the measuring device comprises a tubular body portion, and wherein the method comprises mounting the measuring device on the distal end of the first laparoscopic grasper by extending the jaws of the first laparoscopic grasper when in the closed state through the tubular body portion to releasably mount the measuring device on the first laparoscopic grasper outside the body of the patient. The distal end of the first laparoscopic grasper with the measuring device mounted thereon is introduced through a trocar into the body of the patient adjacent the internal anatomy. Thereafter a length of the internal anatomy is measured via use of the at least one wing.

In accordance with another preferred aspect of the method of this invention the measuring device comprises a tubular body portion, and wherein the method comprises introducing the measuring device by itself through a trocar into the body of the patient adjacent the internal anatomy whereupon the at least one wing automatically pivots to the open state. The measuring device is releasably mounted on a distal end of a first laparoscopic grasper that is extended through a trocar into the body of the patient adjacent the anatomic structure. Once the measuring device is mounted on the distal end of the first laparoscopic grasper a length of the internal anatomy is measured via use of the at least one wing.

### BRIEF DESCRIPTION OF SEVERAL VIEWS OF THE DRAWINGS

FIG. 1 is an isometric view of one exemplary embodiment of a measuring device constructed in accordance with this invention that is configured to be mounted on a standard type manual (non-robotic) laparoscopic grasper including a pair of extendable wings for introduction through a trocar into the abdomen of a patient to measure internal anatomy of the patient, e.g., a section or length of the patient's bowel;

FIG. 2 is an enlarged isometric view of the measuring device shown in FIG. 1;

FIG. 3 is an enlarged top plan view of the measuring device shown in FIGS. 1 and 2;

FIG. 4 is an enlarged side elevational view of the measuring device shown in FIGS. 1 and 2;

FIG. 5 is an isometric view of the measuring device of FIG. 1 shown mounted on a conventional grasper and introduced into the insufflated abdomen of a patient via a conventional trocar;

FIG. 6 is an illustration of use of the measuring device of this invention when mounted on a conventional grasper and being used to measure a length of a patient's bowel by reference to the location of another conventional grasper;

FIGS. 7A-7E are illustrations of sequential steps in the measuring of a section of a patient's bowel by use of the device of this invention mounted on a conventional grasper;

FIG. 8 is an isometric view of alternative embodiment of a measuring device of this invention;

FIG. 9 is an isometric view of another alternative embodiment of a measuring device of this invention;

FIG. 10 is an isometric view of still another alternative embodiment of a measuring device of this invention;

FIG. 11 is an isometric view of yet another exemplary embodiment of a measuring device constructed in accordance with this invention that is configured to be mounted on a robotic laparoscopic grasper or a manual, non-robotic, laparoscopic grasper for introduction through a trocar into the abdomen of a patient to measure internal anatomy of the patient, e.g., a section or length of the patient's bowel, with the grasper shown in FIG. 11 being a manual, non-robotic, laparoscopic grasper;

FIG. 11A is an enlarged isometric view of the distal end of the grasper with the exemplary embodiment of the measuring device of FIG. 11 mounted thereon;

FIG. 11B is an enlarged isometric view, like that of FIG. 11A, but taken from the opposite side of the measuring device shown in FIG. 11A;

FIG. 12 is an isometric view of the exemplary measuring device of FIG. 11, but shown mounted on a robotic laparoscopic grasper for introduction through a trocar into the abdomen of a patient to measure internal anatomy of the patient, e.g., a section or length of the patient's bowel;

FIG. 12A is an enlarged isometric view of the distal end of the robotic grasper of FIG. 12, with the exemplary embodiment of the measuring device of FIG. 12 mounted thereon;

FIG. 12B is an enlarged side view of the robotic grasper of FIG. 12, with measuring device of FIG. 12 mounted thereon;

FIG. 13 is an enlarged isometric view of the exemplary measuring device of FIG. 11, but taken from the distal end thereof;

FIG. 14 is a side elevation view of the exemplary measuring device of FIG. 11;

FIG. 15 is an enlarged sectional view of the exemplary measuring device of FIG. 11 taken along line 15-15 of FIG. 14;

FIG. 16 is an isometric view, partially in section, of the exemplary measuring device of FIG. 11 taken along a plane extending parallel to the longitudinal axis X of the measuring device;

FIG. 17 is an isometric view, partially in section, of the exemplary measuring device of FIG. 11 taken along another plane extending parallel to the longitudinal axis X of the measuring device;

FIG. 18 is a distal end view of the exemplary measuring device of FIG. 11;

FIGS. 19A-19C are illustrations showing sequential steps in the mounting of the exemplary measuring device of FIG. 11 onto a laparoscopic grasper;

FIG. 20 is an isometric view of the measuring device of FIGS. 11 and 12 shown in the process of being removed from the abdomen of a patient by a robotic grasper, like that of FIG. 12, after the measuring device had been utilized to measure a section of the patient's bowel;

FIG. 21 is an isometric view of yet another exemplary embodiment of a measuring device constructed in accordance with this invention and identical to the embodiment shown in FIG. 11, but only having a single wing;

FIGS. 22A-22E are illustrations similar to FIGS. 7A-7E of sequential steps in the measuring of a section of a patient's bowel by use of the exemplary measuring device of FIG. 21;

FIG. 23 is an isometric view of a manual, non-robotic, laparoscopic grasper including a pair of integral measuring wings constructed in accordance with another aspect of this invention for introduction through a trocar into the abdomen of a patient to measure internal anatomy of the patient, e.g., a section or length of the patient's bowel; and

FIG. 23A is an enlarged isometric view of the distal end of the laparoscopic grasper of FIG. 21.

### DETAILED DESCRIPTION OF PREFERRED EMBODIMENTS OF THE INVENTION

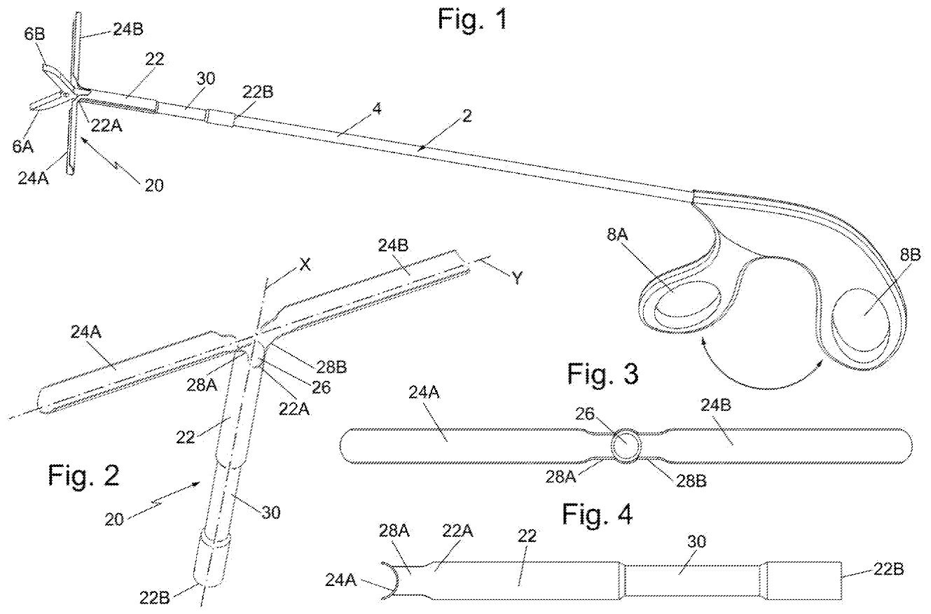

Referring now to the various figures of the drawing wherein like reference characters refer to like parts, there is shown in FIG. 1 one exemplary embodiment of a measuring device 20 of this invention shown mounted on the distal end of a conventional grasper 2. The measuring device when mounted on a grasper can be readily inserted into the body of a patient laparoscopically via a conventional trocar to measure a length of the bowel of the patient. Before describing the details of the measuring device 20, a brief description of the grasper 2 is in order. Thus, as can be seen in FIG. 1, the grasper 2 basically comprises an elongated shaft 4 having a distal end at which a pair of movable jaws 6A and 6B is located. The proximal end of the shaft is connected to a handle having two finger hole handles 8A and 8B. The handles 8A and 8B and the jaws 6A and 6B are coupled together by the shaft 4, in a conventional manner so that squeezing of the finger hole handles together as shown by the double headed arrow in FIG. 1 causes the jaws to pivot from their open state, like shown in FIG. 1, to their closed state. In their closed state the jaws are in engagement and parallel to each other so that anything between the jaws will be grasped by them.

Turning now to FIGS. 2-4 the details of the measuring device will now be described. That device basically comprises an integral unit having a body portion defined by a tubular sidewall 22 and a pair of wings 24A and 24B projecting outwards from the sidewall. The measuring device is preferably formed, e.g., molded as a unit, of a plastic (e.g., Polyether block amide (PEBA)) or some other self-supporting but somewhat resilient material (e.g., Nylon), and is of a constant thickness throughout. That thickness is approximately 0.25 mm, and is sufficiently thin so that the tubular body somewhat flexible and stretchable for reasons to become apparent later. The sidewall 22 includes a distal end 22A and a proximal end 22B, with a central passageway 26 extending between the distal and proximal ends along a longitudinal axis X. The central passageway is configured to receive a distal portion of the shaft of the grasper to releasably mount the measuring device thereon. In particular, as will be described later, a portion of the inner surface of the central passageway 26 will be in frictional engagement with the outer surface of the distal portion of the grasper shaft 4 to mount the measuring device on the shaft at a desired position and to hold it at that position against accidental displacement during use of the measuring device.

As best seen in FIGS. 2 and 4, the wing 24A is a linear member of semicircular cross-section and is connected to the distal end 22A of the tubular body portion 22 by a pivotable hinge 28A, which is preferably a living hinge. The wing 24A in its normal or unbiased state extends along an axis Y which is perpendicular to the longitudinal axis X. The wing 24B is also a linear member of semicircular cross-section and is connected to the distal end of the tubular body portion by a pivotable hinge 28B, which is also preferably a living hinge. The wing 24B is of identical shape and size to the wing 24A and in its normal or unbiased state extends along the axis Y in the opposite radial direction from the wing 24A. Each wing is of a predetermined length, e.g., 5 cm, 10 cm (or any other desired length), measured from its associated living hinge to its distal free end. The living hinges 28A and 28B enable the wings 24A and 24B to be pivoted or flexed thereabout by the application of a bias force to the wings so that the wings move from their normal open position or extended orientation wherein they extend along the perpendicular axis Y, to a closed position or collapsed orientation wherein they extend parallel to each other and parallel to the longitudinal axis X. That feature enables the measuring device 20 when mounted on the grasper 2 to be readily inserted into the body of the patient via a trocar or some other access port.

The tubular sidewall 22 of the measuring device 20 includes a retention structure that constrains the position of the measuring device on the distal end portion of the grasper shaft 4 so that it isn't accidentally displaced from that position during use of the device. The retention structure of the measuring device 20 of FIG. 1 consists of a necked-down intermediate region 30 so that the inner diameter of the passageway 26 thereat is slightly less than the external diameter of the distal end portion of the shaft 4 of the grasper. Moreover, the material from which the measuring device 20 is formed and the thinness of the sidewall making up its tubular body portion 20 renders the necked-down intermediate region 30 slightly stretchable from its normal unbiased state to an expanded state to facilitate the mounting of the measuring device on the grasper. To that end, the grasper's finger holes 8A and 8B are squeezed together to move its jaws 6A and 6B into their closed state. With the jaws of the grasper in their closed state, the measuring device and the grasper are juxtaposed to each other so that the free ends of the closed jaws are aligned with the open proximal end of the passageway 26. The measuring device and the grasper can then be moved relative to each other so that the jaws enter into the passageway 26 and move down it to enter into and pass through the portion of the passageway at the necked down region 30, whereupon the trailing distal end of the grasper shaft enters that passageway portion and causes it to stretch out radially. That action results in the inner surface of that portion of the passageway tightly encircling and frictionally engaging the outer surface of the shaft of the grasper. Notwithstanding that frictional engagement, the measuring device and the grasper can continue to be moved relative to each other until the jaws 24A and 24B clear the distal end 22A of the tubular section 22. At that point the measuring device is at the desired position on the grasper with the wings 24A and 24B being in their normal open (i.e., perpendicular) orientation like shown in FIGS. 1 and 2 and is held in that desired position against accidental displacement by the frictional engagement between tubular body portion and the grasper shaft.

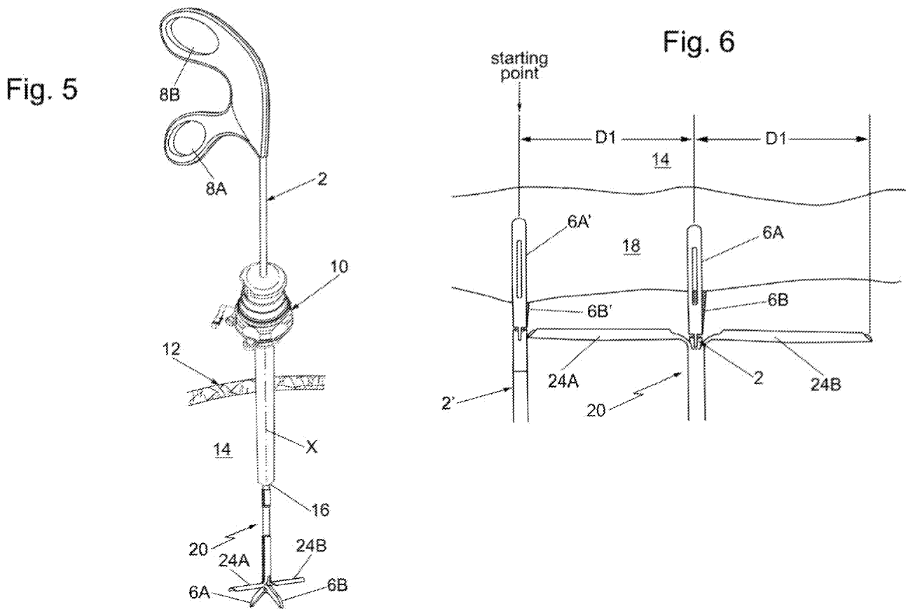

The grasper 2 with the measuring device 20 mounted thereon as just described is now ready for use to measure a section of the patient's bowel laparoscopically by being introduced into the patient's body through a trocar or some other access port. FIG. 5 is an illustration showing that action. In particular, a conventional trocar 10 is shown extending through the abdominal wall 12 of a patient so that the distal end 14 of the trocar is located within the abdominal cavity 16 of the patent adjacent the patient's bowel (which not shown in this illustration). With the trocar in place as just described, the grasper 2 with the measuring device 20 mounted thereon is ready to be inserted through the trocar 10 so that the measuring device will be in the abdominal cavity 14 adjacent the bowel. The wings 24A and 24B of the measuring device 20 can then pivoted from their normally open state or orientation to their closed state or orientation, whereupon they extend parallel to each other and parallel to the longitudinal axis X, with the closed jaws of the grasper interposed between them. The grasper with the measuring device thereon can then be inserted through an access port of a trocar 10 until the wings 24A and 24B of the measuring device are free (past) the distal end 16 of the trocar. Once those wings are free of the distal end of the trocar, they automatically flex outward to assume their normal unbiased, i.e., open state, like shown in FIG. 5. The measuring device is now ready for use to measure a section of bowel.

To accomplish that action a second grasper 2′ is required, like shown in FIG. 6. In particular the second grasper 2′, which is a conventional device constructed like the grasper 2, is extended through an associated trocar (not shown) and its jaws 6A′/6B′ operated to grasp a portion of the bowel 18 between them at the desired starting position for the measurement of a section of the bowel. The position of the grasper 2′ can anywhere the surgeon desires to b e the starting point and can be an anatomical landmark. Once the second grasper 2′ is in place at the starting point, the first grasper 2, with the measuring device 20 mounted thereon can be moved to a first position, whereupon the distal end of the wing 24A is located immediate adjacent (laterally) of the second grasper 2′. The jaws 6A and 6B of the first grasper 2 can then be closed to releasably secure it to the bowel 18 at the first position. The two graspers 2 and 2′ will thus be separated from each other by the distance D1, which is the length of the wings 24A and 24B.

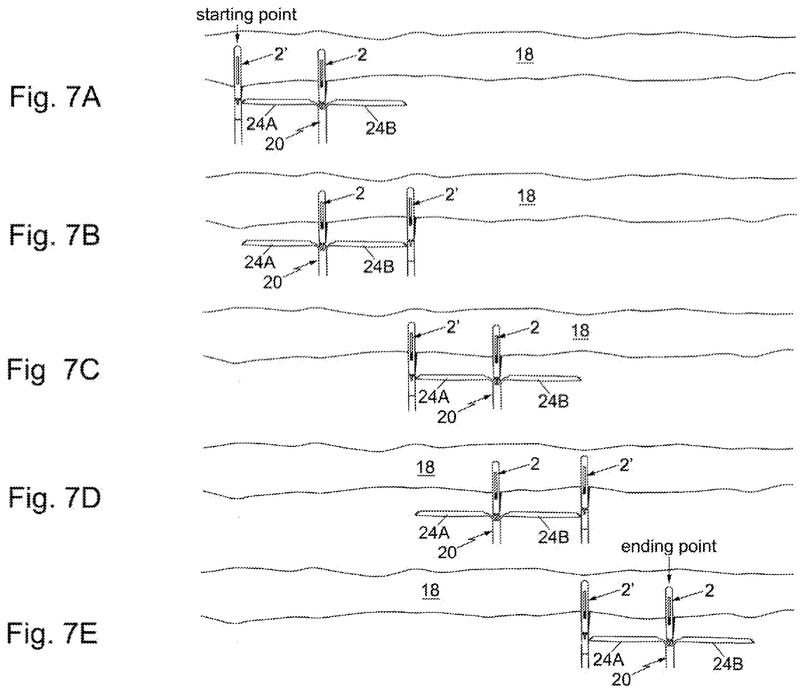

FIG. 7A shows the two graspers 2′ and 2 as just described with respect to FIG. 6, and is the first of a series of illustrations (FIGS. 7A-7E) showing sequential movements or walking of the graspers to measure off a desired length, e.g., 25 cm, of the patient's bowel 18. In particular, after the first grasper 2 has been moved to the first position, the jaws of the second grasper 2′ can be operated to open them to release the second grasper from the bowel at the starting point so that it can be moved or walked to a second position, wherein the second grasper 2′ is located immediately adjacent the distal end of the other perpendicularly extending wing, i.e., wing 24B. Once in that second position the jaws of the second grasper 2′ can be closed to grasp the bowel at that second position like shown in FIG. 7B. Once that has been accomplished, the jaws of the first grasper 2 can be opened to release those jaws from the bowel at the first position and the first grasper 2 then moved or walked to a third position wherein the distal end of one of its perpendicularly extending wings, i.e., wing 24A, is located immediate adjacent the second grasper 2′ as shown in FIG. 7C. The jaws of the first grasper can then be operated to close them and thereby grasp the bowel at that third position. Once that has occurred the jaws of the second grasper 2′ can be operated to open them to release the second grasper from the bowel at the second position so that it can be moved or walked to a fourth position, wherein the second grasper is located immediately adjacent the distal end of the perpendicularly extending wing 24B and then its jaws operated to close them and thus grasp the bowel at that fourth position like shown in FIG. 7D. Once that has been accomplished, the jaws of the first grasper 2 can be opened to release those jaws from the bowel at the third position and the first grasper 2 then moved or walked to a fifth position wherein the distal end of one of its perpendicularly extending wings, i.e., wing 24A, is located immediate adjacent the second grasper 2′ as shown in FIG. 7E. The jaws of the first grasper can then be operated to close and thereby grasp the bowel at that fifth position. In this exemplary series of movements of the graspers the fifth position is the desired ending point for the measurement of bowel.

Since each of the wings is of a length D1, if that length is 5 cm, when the first grasper 2 has been walked as just described to the fifth position a total of 25 cm will have been measured off by the successive incremental movements of those graspers. Accordingly, the 25 cm of the bowel between the starting point and the ending point can then either be bypassed or used as the total length for the common bowel channel. If a section of bowel greater than 25 cm is desired to be measured additional incremental walking movements of the graspers can be accomplished to measure off the desired section of bowel. If a section of bowel less than 25 cm is desired to be measured off, the above described method could be carried out stopping at whatever position is the desired end point of the measured bowel section. It should be readily apparent to those skilled in the art that the measuring device of this invention can make use of wings which are shorter than 5 cm or longer than 5 cm, depending upon the application. In any case all that the user of the measuring device 20 has to do is to count the number of movements of the grasper on which the measuring device is mounted to ensure that the desired length of the bowel has been measured.

It should be noted that the method of measuring the desired length of bowel as just described is somewhat similar to the current prior-art technique of measuring bowel without a length indicating device. That prior-art technique entails the surgeon “walking” two graspers in an overlapping sequential fashion along the bowel. However, by using the measuring device of the subject invention on one of the graspers the same overlapping walking method can be used, but the surgeon will know with certainty where to grab the bowel for each increment as indicated by the wings of the measuring device, utilizing the left arm 24A and the right arm 24B sequentially.

Once the measurement of the bowel has been accomplished using the measuring device and method of this invention the first grasper with the measuring device thereon can be withdrawn from the patient's abdominal cavity 14 by merely pulling on the grasper 2 by its handle to retract the grasper. That retraction action will bring the wings 24A and 24B of the measuring device into engagement with the distal end 16 of the trocar 10, whereupon continued retraction will cause the wings to pivot from their open state (i.e., perpendicular orientation) to their closed state (parallel orientation), whereupon they will enter and pass through the trocar until they reach the proximal end of the trocar and exit the trocar. Once they are free of the proximal end of the trocar the wings will automatically flex back to their open (perpendicular) state. The measuring device can then be removed from the shaft of the grasper to dispose of the measuring device.

It should be pointed out at this juncture that the exemplary measuring device 20 as described above is exemplary of various devices that can be constructed in accordance with this invention. Thus, various modifications can be made to the measuring device within the scope of this invention. For example, various other possible retention features can be implemented in the device to hold the device to the shaft of the grasper in a manner that allows ease of installation while not allowing movement during surgery. Three of such retention features are a retention ring, expansion slits, and a spring.

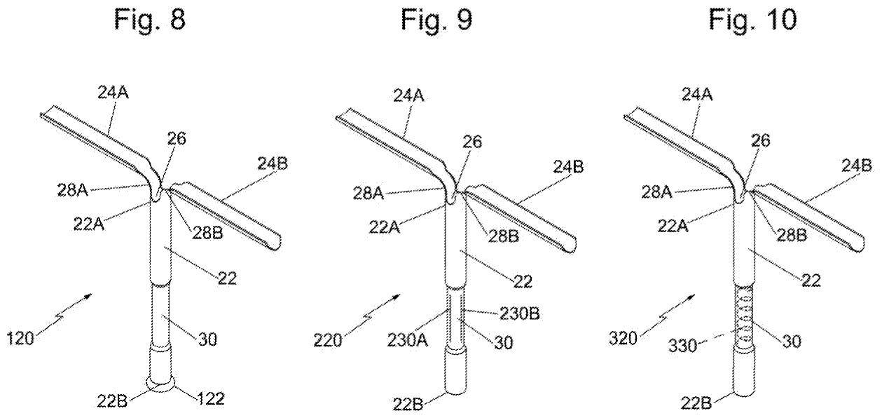

For example in FIG. 8 there is shown an alternative measuring device 120, constructed in accordance with this invention. The measuring device 120 is identical in all respects to the measuring device 20 except that it includes an additional retention structure in the form of a retention ring 122. In the interest of brevity the common features of construction of the measuring device 120 with the measuring device 20 will be given the same reference numbers, and the details of their construction, arrangement and operation will not be reiterated. The retention ring 122 is a rubber or rubber-like (elastic) ring installed at the proximal end 22B of the tubular body section 22 to help retain the device on the shaft of the grasper 2. In this regard the ring 122 provides additional friction for holding the measuring device at the desired position on the shaft of the grasper.

In FIG. 9 there is shown another alternative measuring device 220, constructed in accordance with this invention. The measuring device 220 is identical in all respects to the measuring device 20 except that it includes a means for facilitating the expansion of the necked down section of the tubular body portion. In the interest of brevity the common features of construction of the measuring device 220 with the measuring device 20 will be given the same reference numbers, and the details of their construction, arrangement and operation will not be reiterated. In particular, the means for facilitating the expansion of the necked down section of the tubular body portion is in the form of one or more elongated slits extending longitudinally along the longitudinal axis X in the necked down section 30 of the tubular body portion 22. In FIG. 9 two such slits 230A and 230B are shown. The slits facilitate stretching of the necked down section of the tubular body portion to facilitate the mounting of the measuring device 220 onto the distal end of shaft 4 of the grasper 2.

In FIG. 10 there is shown still another alternative measuring device 320, constructed in accordance with this invention. The measuring device 320 is identical in all respects to the measuring device 20 except that it includes a spring in the necked down section of the tubular body portion. In the interest of brevity the common features of construction of the measuring device 320 with the measuring device 20 will be given the same reference numbers, and the details of their construction, arrangement and operation will not be reiterated. To that end, as can be seen clearly in FIG. 10 a helical spring 330 is located within the necked down section 30. The helical spring 330 is configured to provide additional friction for holding the measuring device 320 in place on the distal end portion of the shaft 4 of the grasper 2 by constricting the necked down portion of the device to the shaft of the grasper.

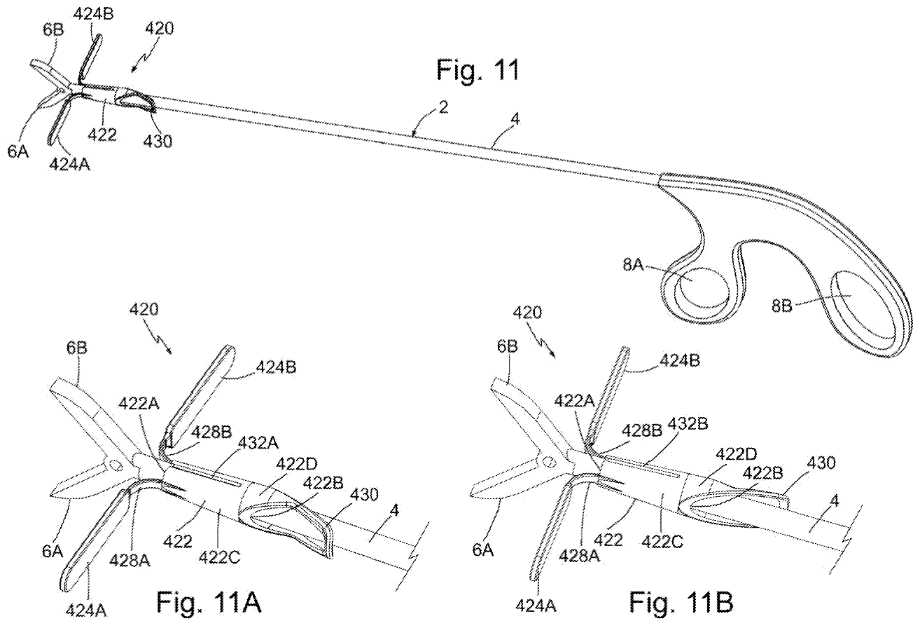

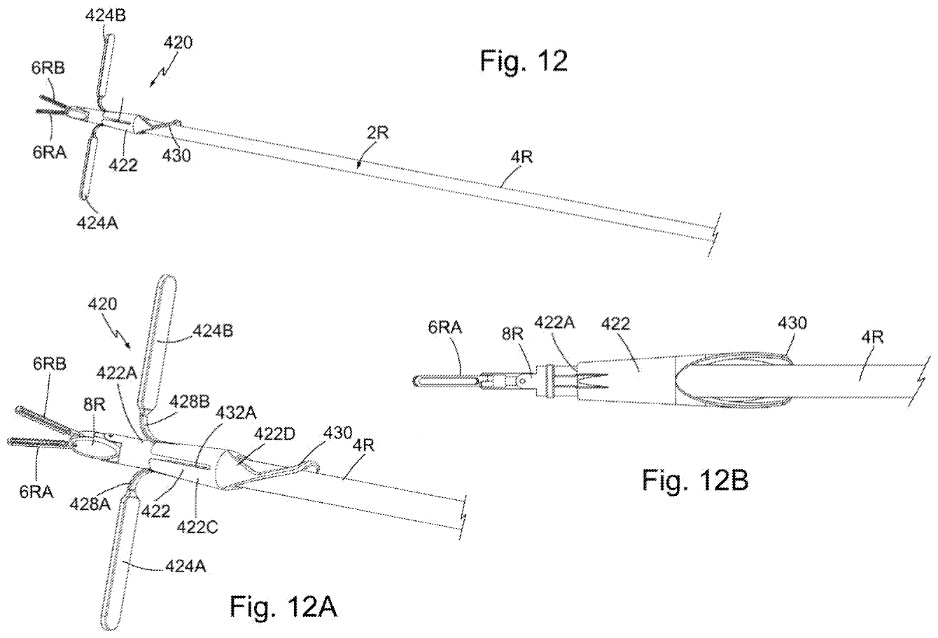

Turning now to FIG. 11, there is shown yet another measuring device 420 constructed in accordance with this invention. The measuring device 420 offers an advantage over the embodiments of the measuring devices 20, 120, 220 and 320 in that has wider applicability, e.g., it can be used on a robotic laparoscopic grasper, e.g., an 8.4 mm diameter robotic grasper (like the grasper 2R shown in FIG. 12), or by scaling it so that it can be used on a standard, e.g., 5 mm diameter, manually operable laparoscopic grasper (like the grasper 2 shown in FIG. 1).

Before describing the details of the measuring device 420 a brief description of the robotic grasper 2R is in order. To that end, as can be seen in FIGS. 12-12B, the grasper 2R basically comprises an elongated shaft 4R having a distal end portion at which a pair of movable jaws 6RA and 6RB is located. The jaws are coupled to a robotic linkage 8R interposed between the distal end of the shaft 4R and the jaws. The proximal end of the shaft 4R is connected to a robotic mechanism (not shown) which is under computer control, so when operated it causes the actuator mechanism 8R to pivot the jaws 8RA/8RB from their open state, like shown in FIG. 12, to their closed state. In their closed state the jaws are in engagement and parallel to each other so that anything between the jaws will be grasped by them.

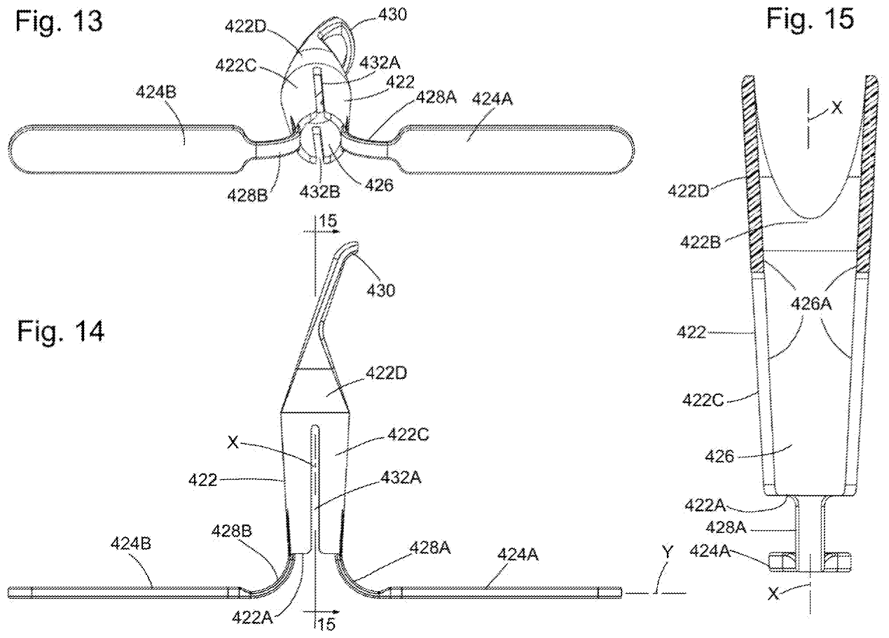

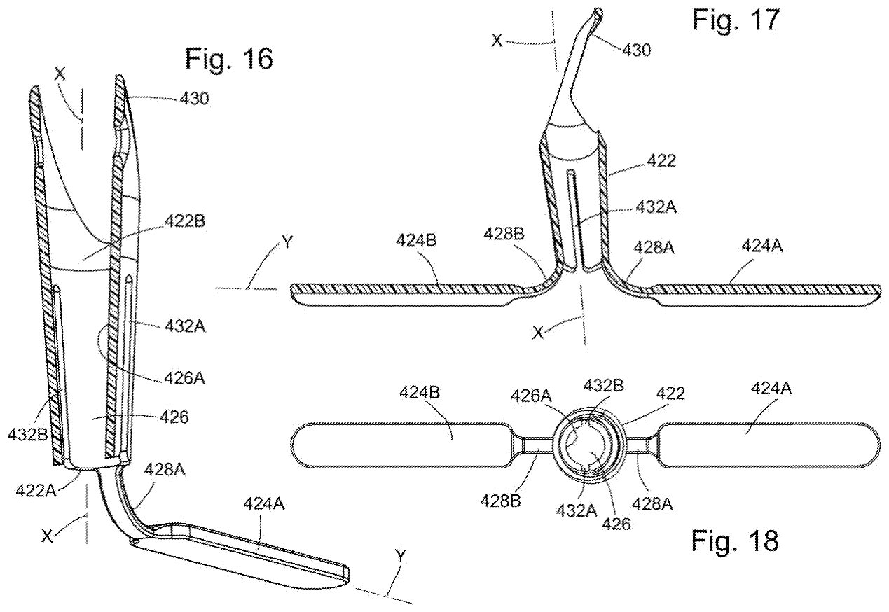

The measuring device 420 is similar in many respects to the measuring devices 20, 120, 220 and 320 in that it basically comprises an integral unit having a body portion in the form of a tubular sidewall 422 from which a pair of wings 424A and 424B project outwards. The sidewall 422 includes a distal end 422A and a proximal end 422B, with a central passageway 426 extending between the distal and proximal ends along a longitudinal axis X. As best seen in FIGS. 13-17 a front section 422C of the sidewall 422 that is contiguous with the distal end 422A tapers downward in the distal direction from approximately the middle of the sidewall. A rear or tail section 422D of the sidewall is contiguous with proximal end of the front section 422C. The rear or tail section 422D is in the form of a loop 430. As best seen in FIGS. 15 and 16 the inner surface 426 of the central passageway tapers downward in the distal direction, with the open end of the central passageway at the distal end 422A having an inside diameter which is less than the outside diameter of the shaft of the grasper 2 or 2R. The proximal end 422B of the passageway 426 is open and has an inside diameter greater than the outside diameter of the shaft of the grasper 2 or 2R. A pair of linear slots 432A and 432B extend from the distal end 422A of the tubular sidewall to approximately the proximal end of the front section 422C and are located diametrically opposite each other.

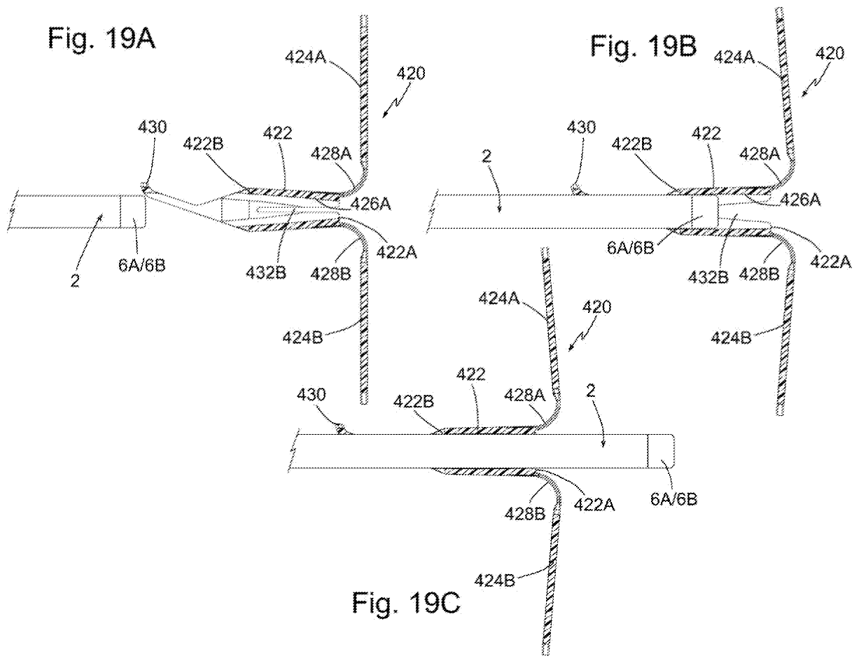

The central passageway 426 is configured to receive a distal portion of the shaft of the grasper on which the measuring device 420 is to be releasably mounted. A portion of the central passageway 426 forms a retention structure to hold the measuring device in place on the grasper at a desired position so that it is resistant to displacement from that desired position. That retention structure constitutes the tapering inner surface 426A of the passageway 426 in the front section 422C which is configured to frictionally engage a peripheral portion of the shaft of the laparoscopic grasper. In particular, as shown in FIG. 19A the jaws 6A/6B at the distal end of the laparoscopic grasper 2 are introduced into the open proximal end of the central passageway with the jaws 6A and 6B of the grasper in their closed position or state, like that described above with respect to the measuring devices 20, 120, 220, and 320. Further insertion of the grasper 2 into the passageway 426 causes the tips of the closed jaws to ride or slide along the tapering inner surface 426A of the central passageway. When the tips of the jaws reach a point on the tapering inner surface of the central passageway where the inside diameter of the passageway at that point is equal to the outside diameter of those tips the pair of slots 432A and 432B will begin to splay apart as shown in FIG. 29B. That action enables the tips of the jaws to pass that point with the inner surface of the portion of the passageway contiguous with the distal end thereof tightly encircling and frictionally engaging the outer surface of the shaft of the grasper. Notwithstanding that frictional engagement, the measuring device 420 and the grasper 2 can continue to be moved relative to each other until the jaws 6A and 6B clear the open distal end 422A of the tubular section 422 like shown in FIG. 19C. At that point the measuring device 420 is at the desired position on the grasper 2 with the wings 424A and 424B being in their unbiased orientation, whereupon they extend transversely to the longitudinal axis X. The measuring device will be held in that desired position against accidental displacement by the frictional engagement between retention structure of the tubular body portion and the grasper's shaft.

As best seen in FIGS. 11A and 11B, the wings 424A and 424B are identical structures, with each wing being an elongated linear member having a generally flat cross-section. The wing 424A is connected to the distal end 422A of the tubular body portion 422 by a living hinge 428A, with the wing 424B connected to the distal 422A of the tubular body portion 422 by a living hinge 428B. As best seen in FIGS. 14-17 the wing 424A in its normal or unbiased state extends along an axis Y which is perpendicular to the longitudinal axis X. The wing 424B in its normal or unbiased state extends along an axis Y which is perpendicular to the longitudinal axis X and in the opposite radial direction from the wing 424A. Each wing is of a predetermined length, e.g., 5 cm, 10 cm (or any other desired length), measured from its associated living hinge to its distal free end. The living hinges 428A and 428B enable the wings 424A and 424B to be pivoted or flexed thereabout by the application of a bias force to the wings so that the wings move from their normal open position or state wherein they extend along the perpendicular axis Y, to a closed position or state wherein they extend parallel to each other and parallel to the longitudinal axis X. That feature enables the measuring device 420 when mounted on a manual laparoscopic grasper 2 to be readily inserted into the body of the patient via a trocar or some other access port.

Once mounted on the grasper 2 and inserted into the body of the patient the grasper with the measuring device mounted thereon can be used in the same manner as the measuring devices 120, 220, and 320 described earlier to measure a section or length of an internal anatomic structure, e.g., the patient's bowel.

The mounting of the measuring device 420 on a robotic laparoscopic grasper 2R is different from the manner in which it is mounted on a manual grasper 2. In particular, the measuring device 420 is designed to be installed onto the shaft 4R of the robotic laparoscopic grasper inside of the patient's body instead of outside the patient's body, as is the case of mounting the measuring device 420 on a manual grasper 2.

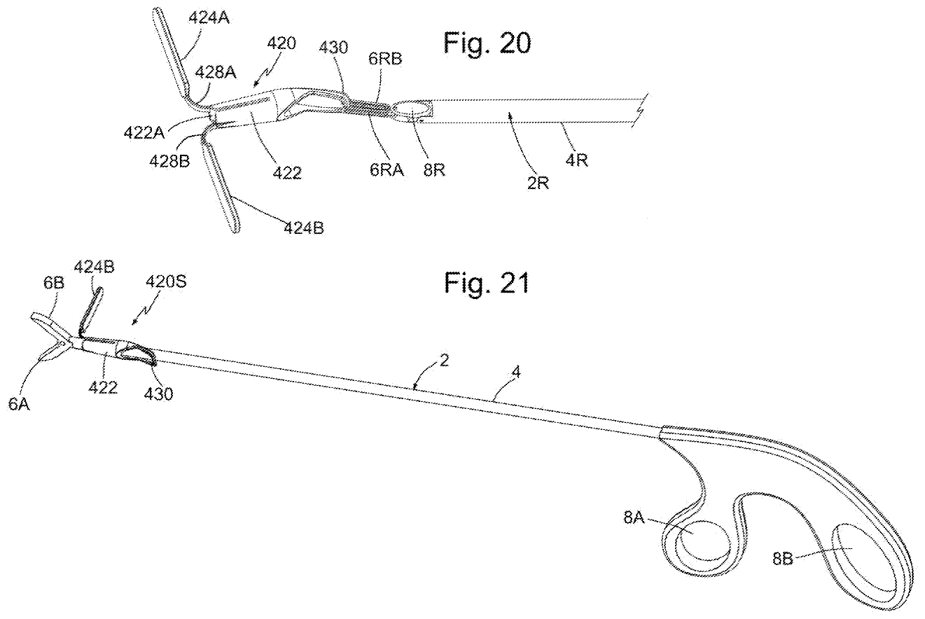

The procedure of mounting the measuring device 420 on a robotic grasper 2R can be accomplished by use of a first robotic grasper (which will be referred to hereinafter as the “introducing grasper”) pushing the measuring device 420 through a trocar into the patient's abdomen. To accomplish that end, the wings 424A and 424B are pivoted to their closed position and once in that position they are introduced into the open outside (proximal) end of a trocar that is extended into the patient's abdomen. Once the measuring device 420 is inside the trocar, the introducing grasper 2R can grasp the tail section 422D of the device at the open outside end of the trocar to push the measuring device through the trocar and into the patient's abdomen. Once inside the patient's abdomen and free of the trocar, the wings 424A and 424B of the measuring device 420 will automatically assume their open position, so that the measuring device is now ready to be mounted on the distal end of a second robotic grasper 2R (which will be referred to hereinafter as the “measuring grasper”) to measure a section of the patient's bowel. To that end, as shown in FIG. 20 the loop 430 of the measuring device 420 can then be grasped by either the jaws of the introducing grasper 2R or some other robotic grasper that is extended into the patient's abdomen. Once that has been accomplished the jaws 6RA/6RB of the measuring grasper 2R can be moved to their closed state. With the jaws closed they can be introduced into the open proximal end 422B of the measuring device by the introducing grasper pulling the measuring device with respect to the measuring grasper. Alternatively, the introducing grasper can hold the measuring device 420 stationary and the measuring grasper can be moved with respect to the measuring device to push the closed jaws into the open proximal end 422B of the measuring device. The introducing grasper (or some other grasper whose jaws are holding the loop 430 of the measuring device 420) and the measuring grasper can be moved with respect to each other so that the closed jaws of the measuring grasper ride or slide down the tapering central passageway 426 and out through the open distal end 422A to mount the measuring device onto the measuring grasper in a similar manner as described above with respect to the mounting of the measuring device 420 on the manual grasper 2. Once mounted on the distal end of the measuring grasper the measuring device can be used to measure the anatomic structure, e.g., the bowel, in the same manner as described above.

After the measuring procedure has been accomplished, the measuring device 420 can be removed from the distal end of the measuring grasper 2R as follows. The jaws of the measuring grasper on which the measuring device is mounted are moved to their closed position or state. Once in that state the jaws of another robotic grasper 2R (hereinafter called the “removal grasper”) can grasp the loop 430 of the measuring device to hold the measuring device 420 in place, whereupon the measuring grasper 2R can be moved in the proximal direction so that its closed jaws are pulled through the central passageway of the measuring device and out its open proximal end 422B. Once that has been accomplished the measuring grasper will be free of the measuring device, so that the measuring device can be removed from the abdomen of the patient. That action is accomplished by the removal grasper 2R, whose jaws are grasping the loop 430 of the measuring device 420, being retracted through the trocar in which the removal grasper extends, whereupon the loop of the measuring device enters into the open distal end of that trocar. Continued pulling of the removal grasper in the proximal direction draws further portions of the measuring device into the trocar, whereupon the wings 424A and 424B engage the distal end of the trocar to pivot the wings to their closed position or state to enter into the distal end of the trocar. Continued pulling of the removal grasper in the proximal direction will draw the measuring device out of the trocar, and hence out of the patient's abdomen.

It should be pointed out at this juncture that the measuring device 420 like the other measuring devices discussed previously is preferably formed, e.g., molded as a unit, of a plastic (e.g., Polyether block amide (PEBA)) or some other self-supporting but somewhat resilient material (e.g., Nylon). The thickness of the body of the device varies from 1-1.2 mm and the wings of the device are approximately 1.78 mm thick.

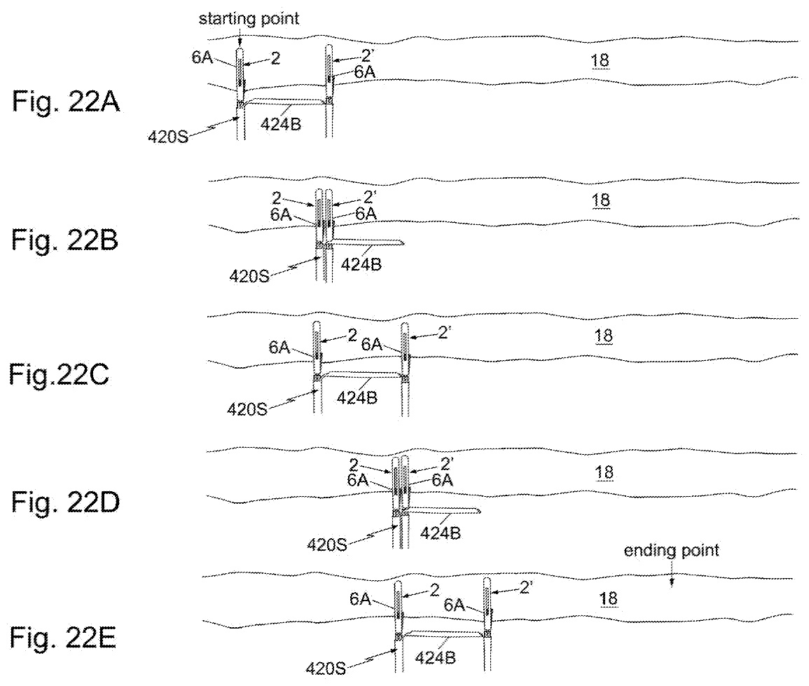

While all of the embodiments of the measuring devices described heretofore have included a pair of extendable/collapsible wings, this invention is broader than that. Thus, this invention contemplates measuring devices making use of only a single extendable/collapsible wing. One such exemplary embodiment 420S of that type of measuring device is shown in FIG. 21. The measuring device 420S is identical in construction to the measuring device 420 as described above, except that it only includes a single extendable/collapsible wing 424B. Thus, in the interest of brevity the features of the measuring device 420S which are common to the features of the measuring device 420 will be given the same reference numbers and the details of their construction and operation will not be reiterated. Moreover, the introduction of the measuring device 420S into the body of the patient and the removal of that measuring device from the body of the patient is identical to that described above. However, owing to the fact that the measuring device 420S only includes a single extendable/collapsible wing the method of using it to measure a section of the internal anatomy of a patient, such as the patient's bowel, is somewhat different. That method of use or operation will now be described with reference to the illustrations in FIGS. 22A to 22E and basically entails the following actions.

The grasper 2 with the measuring device 420S mounted thereon is introduced into the patient's body through a trocar or some other access port in the same manner as described earlier with respect to the measurement of the patient's bowel by the measuring device 20 on the grasper 2. In particular, the grasper 2 with the measuring device 420S thereon (and referred to hereinafter as the “first grasper”) is inserted through an access port of a trocar 10, like that described above with respect to the measuring device 20, whereupon the force applied to the collapsible/extendable wing 424B of the measuring device 420S by the engagement of its wing with the access port of the trocar will pivot the wing to its closed or collapsed state, so that it can pass into and through the trocar into the patient's abdomen. Once the wing is free (past) the distal end 16 of the trocar it's living hinge 428B automatically flexes or pivots the wing outward so that the wing assumes its normal unbiased, i.e., open state, like shown in FIG. 22A. The first grasper 2 with measuring device 420S thereon is now ready for use to measure a section of bowel. To accomplish that end the jaws 6A and 6B of the grasper 2 are opened and the first grasper moved to the starting point to grasp a portion of the bowel 18 at the starting point like shown in FIG. 22A. A second grasper 2′, without a measuring device on it, is extended through an associated trocar (not shown) and its jaws 6A′/6B′ operated to grasp a portion of the bowel 18 at the location of the tip (distal end) of the extended wing 424B of the first grasper 2 like shown in FIG. 22A. The two graspers 2 and 2′ will thus be separated from each other by the distance D1. Once the second grasper 2′ has grasped the bowel at the position of the tip of the wing 424B, the jaws of the first grasper 2 are opened to release those jaws from the bowel, whereupon the first grasper is moved or walked down the bowel toward to desired ending point to a position immediately adjacent the second grasper 2′ so that the tip of its extending wing 424B faces towards the ending point as shown in FIG. 22B. The jaws of the first grasper 2 can then be operated to close them and thereby grasp the bowel at that position. Once that has occurred the jaws of the second grasper 2′ can be operated to open them to release the second grasper from the bowel so that it can be moved or walked down the bowel in the direction of the ending point to the position wherein the second grasper 2′ is located immediately adjacent the distal end of the extending wing 424B and then its jaws operated to close them and thus grasp the bowel at that position like shown in FIG. 22C. Once the second grasper 2′ has grasped the bowel at the position of the tip of the wing 424B, the jaws of the first grasper 2 are opened to release those jaws from the bowel, whereupon the first grasper 2 is moved or walked down the bowel toward to desired ending point to a position immediately adjacent the second grasper 2′ so that the tip of its perpendicularly extending wing 424B faces or extends towards the ending point as shown in FIG. 22D. Once that has occurred the jaws of the second grasper 2′ can be operated to open them to release the second grasper from the bowel so that it can be moved or walked down the bowel in the direction of the ending point to the position wherein the second grasper 2′ is located immediately adjacent the distal end of the perpendicularly extending wing 424B and then its jaws operated to close them and thus grasp the bowel at that position like shown in FIG. 22D. This shuttling procedure can then be repeated as many times as necessary, with the user counting the number of movements of the first grasper 2, until the desired section of the bowel has been measured, i.e., the ending point reached by either grasper as the case may be.

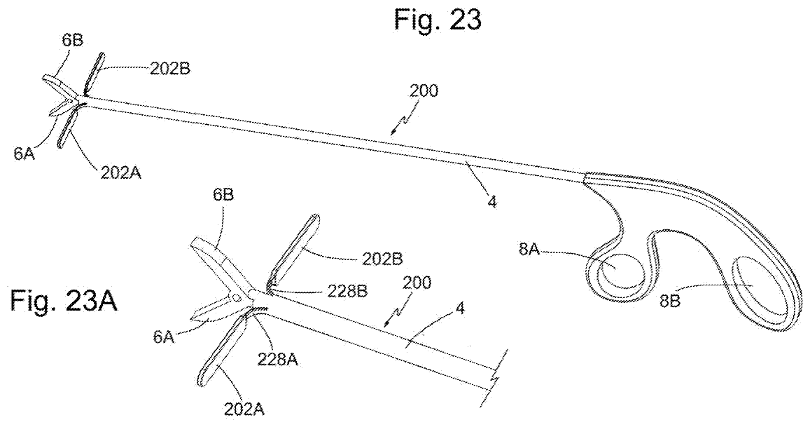

It should also be pointed out that any of the measuring devices of this invention need not be a separate unit configured for releasable mounting on a laparoscopic grasper. Rather, the measuring device can itself be integrated into the grasper, i.e., form a portion of either a manual grasper or a robotic grasper. For example, FIGS. 23 and 23A show a manual grasper 200 with an integrated measuring mechanism constructed in accordance with this invention. The grasper 200 is similar in construction to the grasper 2, except that the shaft 4 of the grasper 200 includes a pair of wings 202A and 202B. Each of the wings is constructed similarly to the wings 424A and 424B. The wings 424A and 424B are connected to the distal end portion of the shaft 204 of the grasper immediately proximally of the jaws 6A and 6B by living hinges 228A and 228B, respectively.

Use of the integrated laparoscopic grasper to measure the bowel or other internal anatomic structure can be accomplished in the same manner as discussed above with respect to FIGS. 6-7E. If the integrated laparoscopic grasper includes only a single extendable/collapsible wing, as is contemplated by this invention, it can be used to measure the bowel or other internal anatomic structure in the same manner as discussed above with respect to FIGS. 22A-22E.

As should be appreciated by those skilled in the art from the above description of the measuring devices and their methods of use, the subject invention provides increased accuracy for surgeons who do not currently measure bowel increments, reducing complications and/or reoperations. Moreover, the subject invention decreases procedure time for surgeons who currently measure the bowel. Further still the flexible wings do not impede current surgical technique or cause damage to bowel or other soft tissue. It should be pointed out at this juncture and should be appreciated by those skilled in the art that while the above description of the structure and use of the measuring devices of this invention have centered on measuring the bowel, that is merely exemplary of various internal anatomic structures that may be measured laparoscopically using the measuring devices and methods of this invention.

Without further elaboration the foregoing will so fully illustrate our invention that others may, by applying current or future knowledge, adopt the same for use under various conditions of service.

We claim:

1. A measuring device for measuring internal anatomy in the body of a patient, said measuring device being configured for mounting on a laparoscopic grasper or being an integral component of the laparoscopic grasper, the laparoscopic grasper having an elongated shaft having a longitudinal axis and pair of grasping jaws located at a distal end of the shaft, said measuring device comprising:
at least one elongated wing having a predetermined length as measured along a longitudinal axis of said at least one elongated wing, said at least one elongated wing being configured for orientation in a state extending generally parallel to said longitudinal axis to enable said at least one elongated wing to be introduced through a trocar or some other port into the body of the patient, said at least one elongated wing also being configured for orientation in a state extending transverse to said longitudinal axis; and
a retention structure for holding said at least one elongated wing on the elongated shaft of the laparoscopic grasper when said measuring device is mounted on the laparoscopic grasper and said at least one elongated wing is located within the body of the patient, whereupon when said at least one elongated wing is in said orientation extending transverse to said longitudinal axis said at least one elongatedwing can be used to measure a length of the internal anatomy of the patient.

2. The measuring device of claim 1, wherein said state extending generally parallel to said longitudinal axis comprises a collapsed or closed state, wherein said state extending transverse to said longitudinal axis comprises a extended or open state, and wherein said measuring device additionally comprises a pivotable hinge biased to normally hold said at least one elongated wing in said open state, said pivotable hinge also being configured to be pivoted in response to an applied force to pivot said at least one elongated wing to said closed state, whereupon when said at least one elongated wing is in said closed state said measuring device can pass through a trocar or some other port into the body of the patient and when said measuring device is in the body of the patient said pivotable hinge automatically pivots said at least one elongated wing to said open state so that said at least one elongated wing can be used to measure a length of the internal anatomy of the patient.

3. The measuring device of claim 2, wherein said measuring device is configured for releasable mounting on the laparoscopic grasper, and wherein said measuring device additionally comprises a body portion including said retention structure configured to receive a distal portion of said laparoscopic grasper to releasably secure said measuring device on the laparoscopic grasper at a desired position.

4. The measuring device of claim 3, wherein said body portion comprises tubular sidewall including a central passageway, a longitudinally extending axis, a distal end and a proximal end, said tubular sidewall having an outer periphery, said central passageway having an inner surface configured to receive a distal portion of the shaft of said laparoscopic grasper to releasably secure said measuring device on the grasper at a desired position, and wherein said at least one wing is located at said distal end of said tubular sidewall.

5. The measuring device of claim 4, wherein said at least one elongated wing comprises a pair of wings, each of which is pivotable between said open state and said closed state, and vice versa, each of said wings of said pair of wings when in said closed state extending parallel to each other and to the longitudinal axis of the shaft to enable the laparoscopic grasper with said measuring device mounted thereon to be introduced through the trocar into the body of the patient, whereupon said pair of wings automatically pivot to said open state, wherein said wings extend in opposite radial directions transverse to said longitudinal axis of said shaft, each of said pair of wings having a predetermined length to serve as measuring tool to enable the user to measure a length of the internal anatomy of the patient.

6. The measuring device of claim 5, wherein the laparoscopic grasper includes a peripheral portion configured to be engaged by a portion of said measuring device, and wherein said central passageway includes a retention structure configured to frictionally engage the peripheral portion of the laparoscopic grasper to hold said measuring device in place on the laparoscopic grasper at a desired position and being resistant to displacement from the desired position, the desired position being such that when said wings are in said open state the grasping jaws of the laparoscopic grasper are exposed and able to open and close as desired.

7. The measuring device of claim 5, wherein each of said wings of said pair of wings is connected to said tubular sidewall by a respective living hinge.

8. The measuring device of claim 5, wherein said body portion and said pair of wings constitute a molded one-piece member.

9. The measuring device of claim 5, wherein said retention structure includes a necked-down section located intermediate said proximal end and said distal end.

10. The measuring device of claim 6, wherein said retention structure includes an elastic ring located at said proximal end, said elastic ring providing additional friction for holding said measuring device at said desired position.

11. The measuring device of claim 6, wherein said retention structure includes a pair of slots in said necked-down section and extending parallel to said longitudinal axis, said slots facilitating stretching of said necked down section.

12. The measuring device of claim 6, wherein said retention structure includes a helical spring in said necked-down section, said helical spring being configured to provide additional friction for holding said measuring device at said desired position.

13. The measuring device of claim 6, wherein said retention structure comprises a front section of said central passageway, said front section tapering in a distal direction and including a pair of slots extending parallel to said longitudinal axis from said distal end of said sidewall to a position adjacent said proximal end of said tubular sidewall.

14. The measuring device of claim 13, wherein an internal diameter of said front section of said central passageway at said distal end is less than an external diameter of the distal end of the laparoscopic grasper, wherein the distal end of the laparoscopic grasper is configured to be introduced into said central passageway from said proximal end of said tubular sidewall and out of said central passageway at said distal end of said tubular sidewall, said pair of slots being configured to splay apart to enable the distal end of the laparoscopic grasper to exit out of said central passageway at said distal end of said tubular sidewall, with portions of said inner surface of said central passageway frictionally engaging portions of the periphery of the laparoscopic grasper.

15. The measuring device of claim 4, wherein said proximal end of said measuring device comprises a loop configured to be grasped by the jaws of a first laparoscopic grasper to enable the measuring device to be mounted on a distal end of a second laparoscopic grasper extending into the body of the patient.

16. The measuring device of claim 15, wherein said loop is configured to be grasped by the jaws of said first laparoscopic grasper by said second laparoscopic grasper to dismount said measuring device from said first laparoscopic grasper.

17. The measuring device of claim 2, wherein said measuring device comprises an integral component of a laparoscopic grasper.

18. A method of laparoscopically measuring internal anatomy of a patient comprising:
providing a first and second laparoscopic grasper, each laparoscopic grasper having a shaft and movable jaws located at a distal end of said shaft;
providing measuring device for releasable mounted on said first laparoscopic grasper, said measuring device having a longitudinal axis and comprising at least one wing having a predetermined length, a proximal end and a distal end, said measuring device being mounted on a distal end of said first laparoscopic grasper with said jaws of said first laparoscopic grasper extending distally beyond said at least one wing;
introducing a second laparoscopic grasper into the body of the patient via a trocar;
moving said first laparoscopic grasper to a first position wherein one of said ends of said at least one wing is located at a starting point and causing said jaws of said first laparoscopic grasper to grasp the internal anatomy at said first position;
moving said second laparoscopic grasper to a second position immediately adjacent one of said ends of said at least one wing and causing said pair of jaws of said second laparoscopic grasper to grasp the internal anatomy at said second position;
causing said jaws of said first grasper to release the internal anatomy at said first position;
moving said first laparoscopic grasper to a third position wherein one of said ends of said at least one wing is located at said second position and causing said jaws of said first laparoscopic grasper to grasp the internal anatomy at said third position; and
if desired repeating said movements of said first and second laparoscopic graspers a desired number of times and counting the number of movements of said first laparoscopic grasper to measure a desired length of the internal anatomy.

19. The method of claim 18, wherein said measuring device comprises a pair of elongated wings whose distal ends extend opposite each other and transverse to said longitudinal axis, wherein when said first laparoscopic grasper is in said first position said distal end of one of said pair of wings is located at said starting point, and said distal end of the other of said pair of wings is located at said second position, and when said first laparoscopic grasper is in said third position said distal end of one of said pair of wings of said first laparoscopic grasper is located at said third position, and said distal end of the other of said pair of wings is located at a fourth position, and wherein said method additionally comprises causing said jaws of said second laparoscopic grasper to be opened to release said second laparoscopic grasper from the internal anatomy and moving said second grasper to said fourth position.

20. The method of claim 18, wherein said measuring device comprises a single elongated wing extending transversely to said longitudinal axis when said first laparoscopic grasper is in said first position, with said proximal end of said at least one wing is located at said starting point and said distal end of said at least one wing being located at said second position, and when said first laparoscopic grasper is in said third position said proximal end of said at least one wing is located at said third position, and said distal end said at least one wing is located at a fourth position, and wherein said method additionally comprises causing said jaws of said second laparoscopic grasper to be opened to release said second laparoscopic grasper from the internal anatomy and moving said second laparoscopic grasper to said fourth position.

21. The method of claim 18, wherein said at least one wing is pivotable between an open state and a closed state, and vice versa, said at least one wing being normally biased into said open state wherein said at least one wing extends transversely to said longitudinal axis, said at least one wing being collapsible to said closed state by a force applied thereto wherein said at least one wing extends parallel to said longitudinal axis, and wherein said method comprises:
causing said at least one wing to be in the closed state to enable the measuring device to be introduced laparoscopically into the body of the patient via a trocar, whereupon once within the body of the patient said at least one wing automatically pivots to said open state.

22. The method of claim 21, wherein said measuring device comprises a tubular body portion, and wherein said method comprises:
mounting said measuring device on said distal end of said first grasper by extending said jaws of said first laparoscopic grasper when in said closed state through the tubular body portion to releasably mount the measuring device on said first laparoscopic grasper outside the body of the patient;
introducing the distal end of said first laparoscopic grasper with said measuring device mounted thereon through a trocar into the body of the patient adjacent said internal anatomy; and
thereafter measuring a length of said internal anatomy via use of said at least one wing.

23. The method of claim 21, wherein said measuring device comprises a tubular body portion, and wherein the method comprises:
introducing said measuring device by itself through a trocar into the body of the patient adjacent said internal anatomy whereupon said at least one wing automatically pivots to said open state;
releasably mounting the measuring device on a distal end of a first laparoscopic grasper that is extended through a trocar into the body of the patient adjacent the anatomic structure; and
once said measuring device is mounted on said distal end of said first laparoscopic grasper measuring a length of said internal anatomy via use of said at least one wing.
